(19) United States
(12) Patent Application Publication
(10) Pub. No.: US 2006/0216698 A1
(43) Pub. Date: Sep. 28, 2006

(54) Fusion expression vector containing the His-tagged Vitreoscilla hemoglobin coding gene

(76) Inventor: Kyung-Won Park, Evanston, IL (US)

Correspondence Address: Kyung-Won Park, Apt 1F, 1237 Elmwood Ave., Evanston, IL, 60202, US

(21) Appl. No.: 11/265,660

(22) Filed: Nov. 3, 2005

(30) Foreign Application Priority Data
Oct. 31, 2003 (KR) 2003-77103

(51) Int. Cl. C12Q 1/70; C07K 14/435; C07H 21/04; C12P 21/06

(52) U.S. Cl. 435/5; 435/69.1; 435/320.1; 435/325; 530/350; 536/23.5

(57) ABSTRACT

The present invention relates to method of providing color to aid in the expression and purification of proteins of E. coli. Vector according to the present invention includes the His-tagged Vitreoscilla hemoglobin (VHb) coding gene upstream of the multiple cloning site. The vector was designed to express target proteins as VHb fusions, which can be purified in one step by affinity chromatography. Due to the color of the heme in VHb, the VHb-fused target proteins have a red color that provides a visual aid for estimating their expression level and solubility. The red color can also be used as a visual marker throughout purification, while the concentration of the fusion protein can be determined by measuring the amount of VHb using carbon monoxide difference spectra. In addition, because of inherently high solubility of VHb, the fusion can increase the solubility of sparingly soluble target proteins. Target proteins can be easily separated from His-tagged VHb due to the presence of a thrombin cleavage site between them.

### RELATED APPLICATIONS

This application claims priority to Korean Patent Application Number 2003-77103, filed on Oct. 31, 2003 and U.S. Provisional Patent Application No. 10/978,653, filed on Nov. 1, 2004.

### FIELD OF THE INVENTION

The present invention relates to cloning systems providing color to aid in the expression and purification of proteins. More particularly, this invention relates to Vitreoscilla hemoglobin (VHb) coding gene producing an easily visible red color when a protein is expressed with VHb as a fusion protein.

### BACKGROUND OF THE INVENTION

Many vectors have been developed for the cloning, expression, and purification of recombinant proteins in E. coli. For example, polypeptides expressed as fusions with maltose binding protein and glutathione S-transferase binding protein can be purified from crude cell lysates by substrate-affinity chromatography (di Guan et al., 1988; Maina et al., 1988; Smith and Johnson, 1988; Guan and Dixon, 1991). Other vectors direct the synthesis of polypeptides without fusion that can be purified by metal affinity chromatography (Studier et al., 1990; Bujard et al., 1987). A major reason for designing fusion expression vectors is that the productivity of fusion proteins is generally higher and more uniform than that of nonfusion proteins. In the case of eukaryotic proteins, the bacterial amino-terminus may stabilize foreign proteins in the bacterial cell (Straus and Gilbert, 1985; Putkey et al., 1985; Bachmair et al., 1986).

One limitation of recent technologies is the absence of any color of the product to aid in visualization of the steps in purification.

The present invention provides cloning systems providing color to aid in the expression and purification of protein and solves the deficiencies currently found in the art.

### BRIEF SUMMARY

In one embodiment, the invention is a DNA vector that includes a fusion protein gene, where the fusion protein gene contains a gene encoding at least 2 consecutive histidines, a Vitreoscilla hemoglobin gene, a gene for a protease recognition site, a multiple cloning site, and a gene for a glycine rich linker.

In another embodiment, the present invention provides a process for expression and purification of a fusion protein encoded by a DNA vector that includes a target protein gene and a globin gene or a gene encoding a fluorescent protein, where the expressed fusion protein produces a color and where the color aids in the purification of the target protein.

In yet another embodiment, the present invention provides a fusion protein containing a target protein and an end segment where the fusion protein further contains at least two consecutive histidines, a globin protein, a protease cleavage site, and a glycine rich linker.

### DESCRIPTION OF THE FIGURES

FIG. 1 is the graphical structure of a recombinant plasmid pKW32.

FIG. 2 is an SDS-PAGE showing the expression and purification of target protein (VHbm3) using a pKW32 recombinant plasmid, affinity chromatography on Ni-NTA resin, and thrombin cleavage.

FIGS. 3A-3B are an SDS-PAGE of the CyoAsol and HIV integrase expressed as fusions to VHb.

FIG. 4 is CO-difference spectra of VHbm3 (A), VHb-CyoAsol fusion protein (B), and VHb-HIV integrase fusion protein (C) purified using pKW32.

FIG. 5 is an Endonuclease assay.

FIG. 6 is SDS-PAGE of the purified HI0305, HI1244, HI1282 and HI1446 proteins using a pKW32 fusion expression vector.

### DESCRIPTION OF INVENTION

The present invention relates to cloning systems providing color to aid in the expression and purification of proteins. VHb (Vitreoscilla hemoglobin encoded by vgb), with its red color, offers a unique method to solve this problem. VHb consists of two identical subunits of 15.7 kD along with two protohemes IX per molecule (Dikshit et al., 1988). The proposed function of VHb is to capture oxygen and facilitate oxygen delivery to the terminal oxidases (Ramandeep et al., 2001; Tsai et al., 1995a; Tsai et al., 1995b; Dikshit et al., 1992; Park et al., 2002). Expression of VHb in heterologous bacteria or yeast often enhances cell density and growth rate, yields of recombinant proteins, production of antibiotics, and bioremediating ability especially under oxygen limiting conditions (Chung et al., 2001; Dikshit et al., 1990; Khosla and Bailey, 1988; Khosravi et al., 1990; Magnolo et al., 1991; Patel et al., 2000).

A preferred embodiment provides a process for fusion protein expression and purification and a measurement of the amount of the target protein using carbon monoxide difference spectra using a fusion protein expression vector pKW32 encoding a Vitreoscilla hemoglobin gene (Vgb). The target protein may be any protein encoded by a gene that can be cloned into the expression vector via the multiple cloning site.

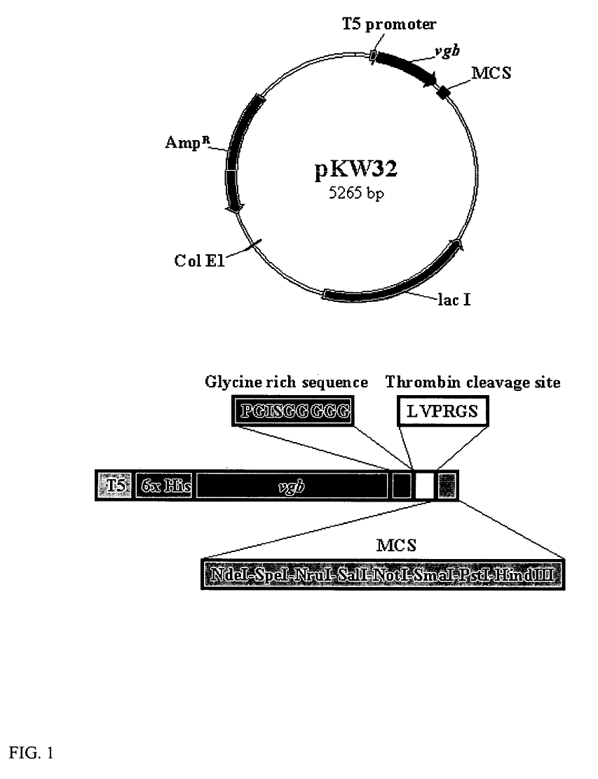

In another embodiment, the DNA vector of the invention contains a protease recognitions site, also referred to herein, as a cleavage site. Examples of suitable protease recognition sites include, but are not limited to, thrombin, Factor Xa and TEV protease. TEV protease recognizes Glu-Asn-Leu-Tyr-Phe-Gln-Ser, which is the seven amino acid consensus sequence. Factor Xa Protease recognizes the amino acid sequence Ile-Glu-Gly-Arg and cleaves the peptide bond C-terminal of the arginine residue. Preferably, a thrombin protease recognition site is present. The present invention is further illustrated in detail using figures. In FIG. 1, the vector contains vgb and a thrombin recognition site. The thrombin recognition site is flanked by a glycine rich sequence. The positions of the ampicillin resistance gene (Ampr), ColE1 replication origin (ColE1), lac repressor (lacI) and multiple cloning sites (MCS) are indicated. Transcription of the fusion genes is controlled by the T5 promoter. The details of the region from the T5 promoter to the MCS are shown beneath the plasmid map in FIG. 1. The preferred embodiment of using fusions to VHb to express and purify foreign proteins gives a red color for the protein in all purification steps. Other visualizable color producing protein encoding genes such as those that code for other proteins in the globin family, such as flavohemoglobin and myoglobin, and those that encode fluorescent proteins, including but not limited to; Green Flourescent Protein (GFP), Yellow Flourescent Protein, and Cyano Flourescent Protein may be used as an alternative to Vgb. The histidine tag, which consists of multiple histidine protein encoding genes, allows for purification of the fusion protein on a nickel-nitrilotriacetic acid (Ni-NTA) matrix in a single chromatography step, this process is well known to those with skill in the art, and the number of histidines used may range from two to twenty or more, but a preferred embodiment contains six. The thrombin cleavage site downstream of vgb, enhanced by the glycine rich linker, allows separation of the VHb from the target proteins after purification, this process is well known to those with skill in the art (Guan and Dixon, 1991).

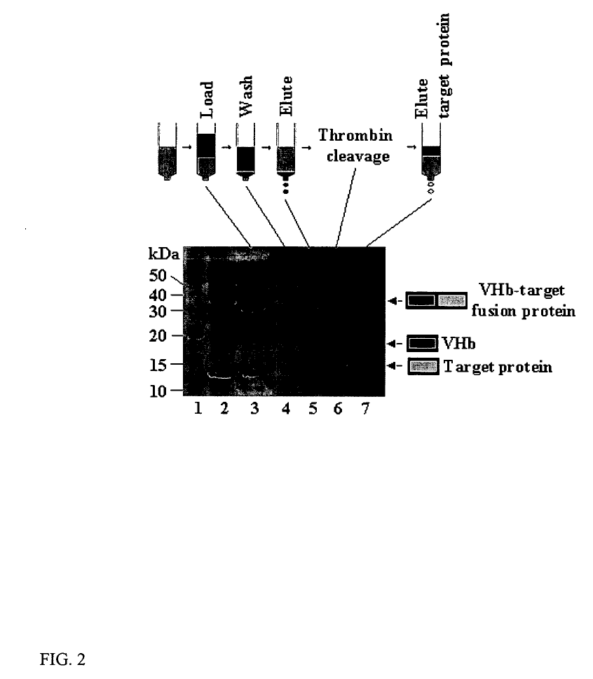

VHbm3, CyoAsol, and HIV integrase were cloned, expressed, and purified using pKW32 to assess the utility of the present invention, however, any protein encoded by a gene that can be cloned into pKW32 can also be expressed and purified. The VHbm3 coding gene was cloned between the NdeI and PstI sites downstream from VHb in pKW32 to produce pKW32:VHbm3. Induced E. coli cultures harboring pKW32:VHbm3 produced the expected 32 kDa VHb-VHbm3 fusion protein which could be purified by a single affinity chromatography step (FIG. 2). FIG. 2 shows SDS-PAGE of the expression and purification process of target protein (VHbm3) using a pKW32 recombinant plasmid, affinity chromatography on Ni-NTA resin, and thrombin cleavage. In FIG. 2, Lane 1 is the molecular weight markers (molecular weights (kDa) shown on the left); lane 2 is the whole cell soluble protein from uninduced cells; lane 3 is the whole cell soluble protein from induced cells; lane 4 is the column flow-through (wash) after loading with sample shown in lane 3; lane 5 is the purified fusion protein after elution; lane 6 is the purified fusion protein after cleavage with thrombin; lane 7 is the purified target protein after rerunning sample in lane 6 on Ni-NTA column.

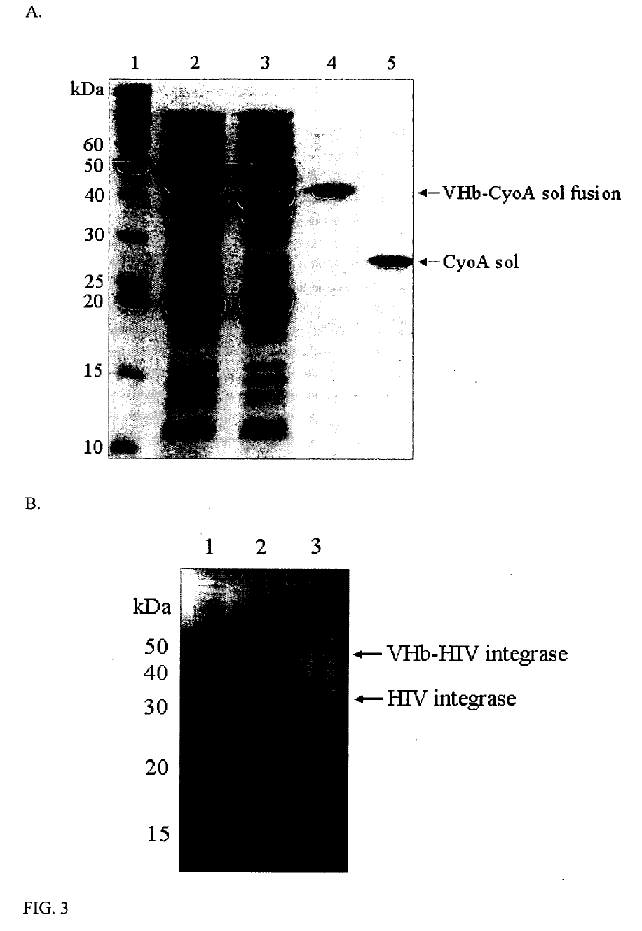

Similarly, the CyoAsol and HIV integrase coding sequences were cloned into the same restriction sites in pKW32 to produce pKW32:CyoAsol and pKW32:HIV integrase. Induced cultures harboring pKW32:CyoAsol and pKW32:HIV integrase gave rise to the expected 43 kDa VHb-CyoAsol and 49kDa VHb-HIV integrase fusion proteins, which could also be easily purified by Ni-NTA chromatography (FIG. 3). FIG. 3 shows an SDS-PAGE of the CyoAsol and HIV integrase expressed as fusions to VHb. In FIG. 3A, Lane 1 is the molecular weight markers (molecular weights (kDa) shown on the left); lane 2 is the whole cell soluble protein from uninduced cells; lane 3 is the whole cell soluble protein from induced cells; lane 4 is VHb-CyoAsol fusion protein purified by Ni-NTA affinity chromatography; lane 5 is CyoAsol fragment after thrombin cleavage and repurification by Ni-NTA affinity chromatography. In FIG. 3B, Lane 1 is the molecular weight markers; lane 2 is the purified VHb-HIV integrase fusion protein; lane 3 is the purified HIV integrase.

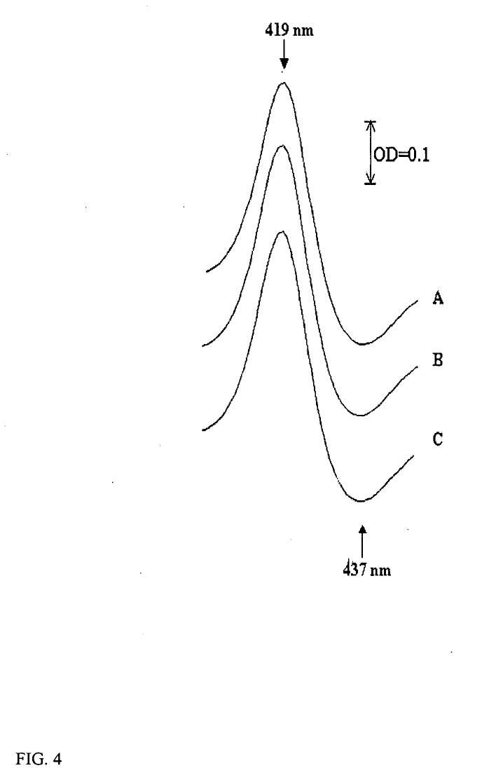

FIG. 4 shows CO-difference spectra of VHbm3 (A), VHb-CyoAsol fusion protein (B), and VHb-HIV integrase fusion protein (C) purified using pKW32. These fusion proteins showed red color and had CO-difference spectra virtually identical to that of native VHb, with absorption maxima in the Soret region at 419 nm and minima at 436 nm (FIG. 4B and C).

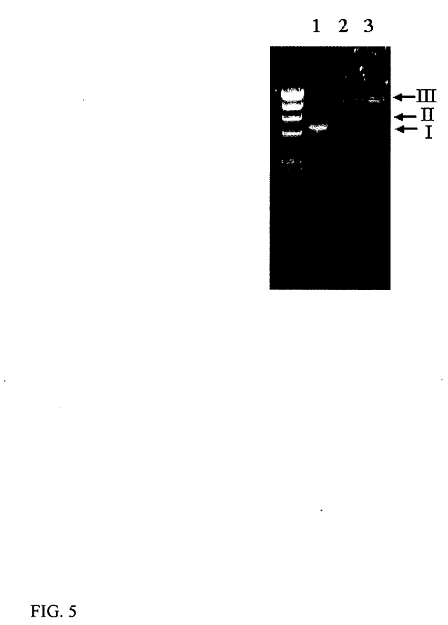

Incubation of the VHb-VHbm3 fusion protein with thrombin led to the production of two fragments, one the His-tagged VHb and the other VHbm3. Similarly, VHb-CyoAsol and HIV integrase fusion proteins were cleaved into two fragments by thrombin and produced His-tagged VHb plus CyoAsol and integrase, respectively. The target proteins in each case were purified away from the His-tagged VHb using the same metal affinity resin used for fusion protein purification. While His-tagged VHb bound to the resin, the VHbm3, CyoAsol, and HIV integrase flowed through the column. Purified VHbm3 also showed an absorption maximum in the Soret region at 419 nm and minimum at 436 nm in its CO-difference spectrum, indicating that target proteins purified using pKW32 can retain native functions (FIG. 4A). This is confirmed by the data in FIG. 5, which shows that HIV integrase purified by this invention system is able to convert supercoiled pJG4-5 to open circular and linear forms; this is essentially identical to activity reported previously for this enzyme using similar substrates (Sherman and Fyfe, 1990; Parissi et al., 2000). In FIG. 5, lane 1 is the native supercoiled pJG4-5; lane 2 is the pJG4-5 incubated with VHb-HIV integrase fusion protein; lane 3 is the pJG4-5 incubated with HIV integrase. I, supercoiled DNA; II, linear DNA; III, open circular DNA. Lefthand lane contains X/HindIII standards.

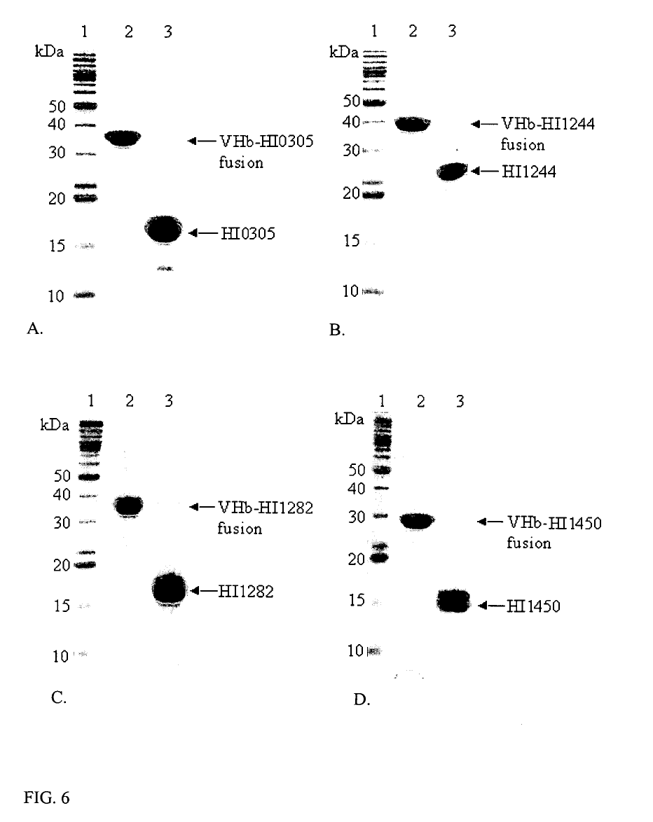

FIG. 6 shows SDS-PAGE of the purified HI0305, HI1244, HI1282 and HI1446 hypothetical proteins from Haemophilus expressed in E. coli using a pKW32 fusion expression vector. In FIGS. 6A-6D, lane 1 is molecular weight markers; lane 2 is the purified VHb-fused HI proteins; Lane 3 is the purified HI proteins. In FIGS. 6A-6D, A is the HI0305; B is the HI1244; C is the HI1282; D is the HI1450.

In particular, the advantages of the preferred embodiment using VHb fusion to express and purify foreign proteins are as follows. First, VHb is not toxic in heterologous bacteria including E. coli. As described above, it is generally even beneficial. Second, the red color of the fusion protein in the cytoplasm and supernatant aids in tracking it through initial production and purification without the need for any special requirements or reagents. Third, since the extinction coefficient for VHb in CO-difference spectra is known, CO-difference spectra of fusion proteins provide a method independent of standard protein assays for quantification of the target protein. Finally, VHb fusion expression may be advantageous in solubilizing many target proteins, which were difficult to express in soluble form using non-fusion expression vectors. Recombinant HIV integrase produced by E. coli has been reported to be insoluble and requires an extensive protocol, including addition of guanidine hydrochloride, for its purification (Bushman et al., 1993; Hickman et al., 1994). In our experiments, however, the HIV integrase was easily purified in soluble form using our standard conditions. Thus, the VHb fusion expression system may be able to facilitate the high-throughput production and purification of a variety of proteins in recombinant systems.

Various modifications and variations of the described methods and compositions of the invention will be apparent to those skilled in the art without departing from the scope and spirit of the invention. The independent claims that follow provide statements of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

### EXAMPLE

The following example further illustrates the invention.

Bacterial Strains and Materials

E. coli strain DH5α was used for the cloning and expression of recombinant genes. Expression vector pKGE (Park et al., 2003), restriction enzymes, Ni-NTA chromatographic resin and PCR Supermix were obtained from Qiagen, New England Biolabs and Life Technologies, respectively. Plasmid preparation, enzymatic manipulation of DNA, bacterial transformation, and PCR amplification were performed according to standard protocols. The template for amplifying the Vitreoscilla VHbm3 gene was supplied by De. Kanak Dikshit, Institute of Microbial Technology, Chandigarh, India (Dikshit et al., 1998); the sequence encoding the soluble region of the A subunit of Vitreoscilla cytochrome bo were isolated and produced in our laboratory (Hwang et al., in press). The full length HIV integrase gene was a gift of Dr. Fred Dyda (NIH).

Construction of Vector pKW32

pKW32 is a derivative of the pKGE expression vector, in which the NdeI site was removed and new multiple cloning sites were added. The next step was to insert vgb with a linker encoding a glycine rich sequence and thrombin cleavage site into pKGE. vgb with a glycine rich linker and thrombin cleavage site was produced by PCR amplification from pNKD1, which contains vgb in vector pBluescript (Dikshit et al., 1998), using upstream primer Sequence ID. #1 5′ GCGCGGGATCCATGTTAGACCAGCAACCATTAACATCATC 3′ and down stream primer Sequence ID #2 5′ GCGCGCATATGTTCACTCCCTCGAGGGACCAAGCCTCCGCCTCCGCCTGATAT TCCTGGTTCAACCGCTTGAGCGTACAAATC 3′. The 5′ end of the downstream primer contains the sequence encoding the thrombin cleavage site followed by the glycine rich sequence. The product was digested with BamHI and NdeI and cloned into pKGE cleaved with same restriction enzymes to create pKW32 (FIG. 1).

Expression and Purification of VHbm3, CyoAsol, and HIV Integrase Using Vector pKW32

Each target gene was amplified by PCR to include an NdeI and a PstI site at either end. Each amplified fragment was digested with NdeI and PstI, cloned into the same sites in pKW32, and expressed in E. coli DH5α. Each log phase culture was induced with 1 mM isopropyl-β-D-thiogalactoside (IPTG) at 37° C. for 4 hours. All subsequent steps were carried out at 4° C. The cell pellet from each 1.5 L culture was resuspended in 50 ml of column buffer (50 mM NaH₂PO₄, pH 8.0; 0.3 M NaCl; 10 mM imidazole) and lysed by incubation for 30 minutes with lysozyme. After centrifugation at 15000 g for 30 minutes, the supernatant was passed through a 5 ml Ni-NTA column at a flow rate of 0.5 ml/min. The column was washed with 20 ml of wash buffer (50 mM NaH₂PO₄, pH 8.0; 0.3 M NaCl; 20 mM imidazole) at the same flow rate. The column was then eluted with 10 ml of elution buffer (50 mM NaH₂PO₄, pH 8.0; 0.3 M NaCl; 250 mM imidazole) at 4° C. Fractions from each step were analyzed by SDS-PAGE using 12% Tris-glycine gels (Laemmli, 1970).

Carbon Monoxide-Difference Spectra

Carbon monoxide difference spectra of purified VHb or VHb fusion proteins were recorded between 400 and 500 nm after bubbling CO into the sample cuvette at room temperature with a Cary Model 210 spectrophotometer as described previously (Dikshit et al., 1988). The concentration of each fusion protein can be calculated from such spectra using the extinction coefficient E(419nm-436nm)=137/mM/cm for the VHb monomer.

HIV Integrase Assay

Endonuclease cleavage of a heterologous DNA substrate was monitored by observing the conversion of supercoiled plasmid DNA to open circular and linear DNA. The typical reaction mixture (20 μl) contained 250 ng of supercoiled pJG4-5 DNA (a 6.5 kb pUC derivative), 50 mM NaCl, 100 mM Tris-HCl (pH 7.5), 10 mM MgCl₂, 0.025% Triton X-100 and the equivalent of 0.03 μl of undiluted VHb-HIV integrase or HIV integrase. The reaction mixtures were incubated for one hour at 37° C. and were stopped by addition of 3 μl of 95% formamide, 20 mM EDTA, 0.05% bromophenol blue stop solution. Samples were electrophoresed on 1% agarose gels for one hour at 150 V at room temperature. All cited references are incorporated herein by reference in their entirety.

### REFERENCES CITED

Bachmair, A., Finley, D., Varshavsky, A., 1986. In vivo half-life of a protein is a function of its amino-terminal residue. Science 234, 179-186.

Bujard, H., Gentz, R., Lanzer, M., Stueber, D., Mueller, M., Ibrahimi, I., Haeuptle, M. T., Dobberstein, B., 1987. A T5 promoter-based transcription-translation system for the analysis of proteins in vitro and in vivo. Methods Enzymol. 155, 416-433.
Bushman, F. D., Engelman, A., Palmer, I., Wingfield, P., Craigie, R.,1993. Domains of the integrase protein of human immunodeficiency virus type 1 responsible for polynucleotidyl transfer and zinc binding. Proc. Natl. Acad. Sci. USA 90, 3428-3432.
Chung, J. W., Webster, D. A., Pagilla, K. R., Stark, B. C., 2001. Chromosomal integration of the Vitreoscilla hemoglobin gene in Burkholderia and Pseudomonas for the purpose of producing stable engineered strains with enhanced bioremediating ability. J Ind Microbiol Biotechnol. 27, 27-33.
di Guan, C., Li, P., Riggs, P. D., Inouye, H., 1988. Vectors that facilitate the expression and purification of foreign peptides in Escherichia coli by fusion to maltose-binding protein. Gene 67, 21-30.
Dikshit, K. L., Webster, D. A., 1988. Cloning, characterization and expression of the bacterial globin gene from Vitreoscilla in Escherichia coli. Gene 70, 377-386.
Dikshit, K. L., Dikshit, R. P., Webster, D. A., 1990. Study of Vitreoscilla globin (vgb) gene expression and promoter activity in E. coli through transcriptional fusion. Nucleic Acids Res. 18, 4149-4155.
Dikshit, R. P., Dikshit, K. L., Liu, Y., Webster, D. A., 1992. The bacterial hemoglobin from Vitreoscilla can support the aerobic growth of Eschericia coli lacking terminal oxidases. Arch. Biochem. Biophys. 293, 241-245.
Dikshit, K. L., Orii, Y., Navani, N., Patel, S., Huang, H. Y., Stark, B. C., Webster, D. A., 1998. Site-directed mutagenesis of bacterial hemoglobin: the role of glutamine (E7) in oxygen-binding in the distal heme pocket. Arch. Biochem. Biophys. 349, 161-166.
Guan, K. L., Dixon, J. E., 1991. Eukaryotic proteins expressed in Escherichia coli: an improved thrombin cleavage and purification procedure of fusion proteins with glutathione S-transferase. Anal. Biochem. 192, 262-267.
Hickman, A. B., Palmer, I., Engelman, A., Craigie, R., Wingfield, P., 1994. Biophysical and enzymatic properties of the catalytic domain of HIV-1 integrase. J. Biol. Chem. 269, 29279-29287.
Hwang, K. W., Kim, S. K., Kim, K. J., Chung, Y. T., Stark, B. C., Webster, D. A., Isolation, Sequencing, and Characterization of the Cytochrome bo Operon from Vitreoscilla. DNA Sequence (in press).
Khosravi, M., Webster, D. A., Stark, B. C., 1990. Presence of the bacterial hemoglobin gene improves alpha-amylase production of a recombinant Escherichia coli strain. Plasmid 24, 190-194.
Khosla, C., Bailey, J. E., 1988. Heterologous expression of a bacterial hemoglobin improves the growth properties of recombinant Escherichia coli. Nature 331, 633-635.
Kim, K. J., Kim, Y. C., Park, K. W., Webster, D. A., Howard, A. J., 2002. Purification, crystallization and preliminary X-ray analysis of the soluble domain of the Na⁺ pumping cytochrome bo quinol oxidase from Vitreoscilla. Acta. Crystallographica. Section D. 58, 1329-1331
Laemmli, U. K., 1970. Cleavage of structural proteins during the assembly of the head of bacteriophage T4. Nature 227, 680-685.
Magnolo, S. K., Leenutaphong, D. L., De Modena, J. A., Curtis, J. E., Bailey, J. E., Galazzo, J. A., Hughes, D. E., 1991. Actinorhodin production by Streptomyces coelicolor and growth of Streptomyces lividans are improved by the expression of a bacterial hemoglobin. Biotechnology 9, 473-476.
Maina, C. V., Riggs, P. D., Grandea, A. G., Slatko, B. E., Moran, L. S., Tagliamonte, J. A., McReynolds, L. A., Guan, C. D., 1988. An Escherichia coli vector to express and purify foreign proteins by fusion to and separation from maltose-binding protein. Gene 74, 365-373.
Parissi, V, Caumont, A. B., de Soultrait, V. R., Calmels, C., Pichuantes, S., Litvak, S., Dupont, C. H., 2000. Selection of amino acid substitutions restoring activity of HIV-1 integrase mutated in its catalytic site using the yeast Saccharomyces cerevisiae. J. Mol. Biol. 295, 755-765.
Park, K. W., Kim, K. J., Howard, A. J., Stark, B. C., Webster, D. A., 2002. Vitreoscilla Hemoglobin binds to Subunit I of Cytochrome bo Ubiquinol Oxidases. J. Biol. Chem. 277, 33334-33337.
Park, K. W., Webster, D. A., Stark, B. C., Howard, A. J., Kim, K. J., 2003. Fusion protein expression system designed to provide color to aid in the expression and purification of proteins in Escherichia coli. Plasmid 50, 169-175.
Patel, S. M., Stark, B. C., Hwang, K. W., Dikshit, K. L., Webster, D. A., 2000. Cloning and expression of Vitreoscilla hemoglobin gene in Burkholderia sp. strain DNT for enhancement of 2,4-dinitrotoluene degradation. Biotechnol. Prog. 16, 26-30.
Putkey, J. A., Slaughter, G. R., Means, A. R.,1985. Bacterial expression and characterization of proteins derived from the chicken calmodulin cDNA and a calmodulin processed gene. J. Biol. Chem. 260, 4704-4712.
Ramandeep, Hwang, K. W., Raje, M., Kim, K. J., Stark, B. C., Dikshit, K. L., Webster, D. A.,2001. Vitreoscilla hemoglobin. Intracellular localization and binding to membranes. J. Biol. Chem. 276, 24781-24789.
Sherman P. A., Fyfe J. A., 1990. Human immunodeficiency virus integration protein expressed in Escherichia coli possesses selective DNA cleaving activity. Proc. Natl. Acad. Sci. USA 87, 5119-5123.
Smith, D. B., Johnson, K. S., 1988. Single step purification of polypeptides expressed in Escherichia coli as fusions with glutathione S-transferase. Gene 67, 31-34.
Straus, D., Gilbert, W., 1985. Chicken triosephosphate isomerase complements an Escherichia coli deficiency. Proc. Natl. Acad. Sci. USA 82, 2014-2018.
Studier, F. W., Rosenberg, A. H., Dunn, J. J., Dubendorff, J. W., 1990. Use of T7 RNA polymerase to direct expression of cloned genes. Methods Enzymol. 185, 60-89.
Tsai, P. S., Hatzimamikatis, V., Bailey J. E., 1995. Effect of Vitreoscilla hemoglobin dosage on microaerobic Escherichia coli carbon and energy metabolism. Biotechnol. Bioeng. 49, 139-150.
Tsai, P. S., Nagely, M., Bailey J. E., 1995. Intracellular expression of Vitreoscilla hemoglobin modifies microaerobic Escherichia coli metabolism through elevated concentration and specific activity of cytochrome o. Biotechnol. Bioeng. 49, 151-160.

What is claimed is:

1. A DNA vector comprising a fusion protein gene, wherein the fusion protein gene comprises a gene encoding at least 2 consecutive histidines, a globin gene or a gene encoding a fluorescent protein, a gene for a protease recognition site, a multiple cloning site, and a gene for a glycine rich linker.

2. The DNA vector of claim 1 wherein said globin gene is a hemoglobin gene.

3. The DNA vector of claim 1 wherein said hemoglobin gene is a Vitreoscilla hemoglobin gene.

4. The DNA vector of claim 1 wherein said glycine rich linker is down stream from said thrombin recognition site.

5. The DNA vector of claim 1 wherein said vector is a plasmid vector.

6. The DNA vector of claim 5 wherein said plasmid vector is pKW32.

7. The DNA vector or claim 1 wherein the multiple cloning site is a restriction site cleaved by one selected from the group consisting of NdeI, SpeI, NruI, SalI, NotI, EcoRI, SmaI, PstgI, and HindIII.

8. The DNA vector of claim 1 wherein the fluorescent-type protein is selected from the group consisting of GFP, YFP, and CFP.

9. A host cell transformed or transfected with the vector of claim 1.

10. The host cell of claim 9 wherein the host cell is a bacterium.

11. The host cell of claim 10 wherein said bacterium is E. coli.

12. The host cell of claim 9 wherein said host cell is a eukaryotic cell.

13. The host cell of claim 12 wherein said eukaryotic cell is selected from the group consisting of a yeast, an insect cell, a mammalian cell.

14. The DNA vector cell of claim 1 wherein the gene for the protease recognition site is selected from the group consisting of a thrombin gene, Factor Xa , and a TEV protease.

15. A process for expression and purification of a fusion protein encoded by a DNA vector comprising a target protein gene, and a globin gene or a gene encoding a fluorescent protein, wherein the expressed fusion protein produces a color and wherein said color aids the purification of the target protein.

16. The process of claim 15 wherein said color is selected from the group consisting of red, green, orange, yellow, or any other fluorescent color.

17. The process of claim 15 wherein said globin gene is a hemoglobin gene.

18. The process of claim 17 wherein said hemoglobin gene is a Vitreoscilla hemoglobin gene.

19. The process of claim 15 wherein said target protein is selected from the group consisting of VHbm3, CyoAsol, and HIV integrase.

20. A fusion protein comprising a target protein and an end segment wherein said fusion protein further comprises at least 2 consecutive histidines, a globin protein, a protease recognition site, and a glycine rich linker.


SEQUENCE LISTING (2 sequences)

SEQ ID NO: 1 - DNA, 40 bp, Vitreoscilla stercoria
gcgcgggatccatgttagaccagcaaccattaacatcatc

SEQ ID NO: 2 - DNA, 83 bp, Vitreoscilla stercoria
gcgcgcatatgttcactccctcgagggaccaagcctccgcctccgcctgatattcctggttcaaccgcttgagcgtacaaatc